(19) United States
(12) Patent Application Publication
(10) Pub. No.: US 2026/0043741 A1
(43) Pub. Date: Feb. 12, 2026

(54) WATER QUALITY DETECTION SYSTEM AND WATER QUALITY DETECTION DEVICE

(71) Applicant: INDUSTRIAL TECHNOLOGY RESEARCH INSTITUTE, Hsinchu (TW)

(72) Inventors: Ming-Chieh CHOU, Kaohsiung City (TW); Cheng Da KO, Tainan City (TW); Tsung-Yu TSAI, Tainan City (TW)

(73) Assignee: INDUSTRIAL TECHNOLOGY RESEARCH INSTITUTE, Hsinchu (TW)

(21) Appl. No.: 19/014,036

(22) Filed: Jan. 8, 2025

(30) Foreign Application Priority Data
Aug. 12, 2024 (TW) 113130172

(51) Int. Cl. G01N 21/64; G01N 33/18

(52) U.S. Cl. CPC G01N 21/64; G01N 33/18; G01N 2021/6497

(57) ABSTRACT

A water quality detection system includes a water detection device, a water-leaving airbag and a gas filling module. The water detection device includes a body, a sensing light source, a fluorescent sensing film, an optical detector and a processor. The sensing light source is disposed in the body and emits sensing light. The fluorescent sensing film is disposed on the surface of the body and generates feedback light in response to the sensing light. The optical detector is disposed in the body and generates a concentration signal in response to the feedback light. The processor determines a concentration of a component to be measured in an aqueous solution according to the concentration signal. The gas filling module is configured to inflate or deflate the water-leaving airbag. The water-leaving airbag is disposed at the body and configured to float the fluorescent sensing film above the liquid surface.

### CROSS-REFERENCE TO RELATED APPLICATIONS

This non-provisional application claims priority under 35 U.S. C. § 119(a) on Patent Application No(s). 113130172 filed in ROC (Republic of China) on Aug., 12 2024, the entire contents of which are hereby incorporated by reference.

### BACKGROUND

### 1. Technical Field

This disclosure relates to a water quality detection system and water quality detection device.

### 2. Related Art

For optical water quality sensors, due to long-term immersion in aqueous solution, the sensor housing and fluorescent sensing film may be worn out, so the water quality sensor needs to be replaced frequently. In particular, barnacles or other microorganisms in seawater or aquaculture water may be easily to adhere to the fluorescent sensing film, and in some water bodies with poor water quality, sensing accuracy of the fluorescent sensing film may decline dramatically within a few days. All of these have led to a decrease in accuracy and increase in maintenance costs for current water quality detection devices.

### SUMMARY

Accordingly, this disclosure provides a water quality detection system and water quality detection device.

According to one or more embodiment of this disclosure, a water quality detection system, which is configured to measure the concentration of a component to be measured in an aqueous solution, includes a water quality detection device and a water-leaving airbag and a gas filling module connected to the water quality detection device. The water quality detection device includes a body, a sensing light source, a fluorescent sensing film, an optical detector and a processor. The sensing light source is disposed in the body and configured to emit sensing light. The fluorescent sensing film is disposed on the surface of the body and configured to receive the sensing light to generate feedback light. The optical detector is disposed in the body and configured to receive the feedback light to generate a concentration signal. The processor is disposed in the body and electrically connected to the optical detector, and configured to determine the concentration of the component to be measured in the aqueous solution according to the concentration signal. The gas filling module is configured to inflate or deflate the water-leaving airbag. The water-leaving airbag is disposed at the body and configured to float the fluorescent sensing film to be above the liquid surface when filled with gas.

According to one or more embodiment of this disclosure, a water quality detection device, which is configured to measure the concentration of a component to be measured in an aqueous solution, includes a body, a sensing light source, a fluorescent sensing film, an optical detector, a processor and an antibacterial light source. The sensing light source is disposed in the body and configured to emit sensing light. The fluorescent sensing film includes a reaction layer and a light-shielding layer, the reaction layer is disposed on a light-transmitting surface of the body, the light-shielding layer is disposed on the reaction layer and is configured to direct contact with the aqueous solution, and the reaction layer is configured to receive the sensing light to generate feedback light. The optical detector is disposed in the body and configured to receive the feedback light to generate a concentration signal. The processor is disposed in the body and electrically connected to the optical detector, and configured to determine the concentration of the component to be measured in the aqueous solution according to the concentration signal. The antibacterial light source is disposed at the body and is configured to emit antibacterial light to one side of the light-shielding layer that is in direct contact with the aqueous solution.

### BRIEF DESCRIPTION OF THE DRAWINGS

The disclosure will become more fully understood from the detailed description given hereinbelow and the accompanying drawings which are given by way of illustration only and thus are not limitative of the disclosure and wherein:

FIG. 1 is a schematic diagram of a water quality detection system according to an embodiment of the disclosure;

FIG. 2 is another schematic diagram of a water quality detection system according to an embodiment of the disclosure;

FIG. 3 is a schematic diagram of a water quality detection system in a sensing state according to another embodiment of the disclosure;

FIG. 4 is a schematic diagram of a water quality detection system according to still another embodiment of the disclosure;

FIG. 5 is a schematic diagram of a water quality detection system according to yet another embodiment of the disclosure;

FIG. 6 is a schematic diagram of a water quality detection device according to an embodiment of the disclosure;

FIG. 7 is another schematic diagram of the water quality detection device according to the embodiment of FIG. 6;

FIG. 8 is a schematic diagram of the antibacterial light source of the water quality detection device according to the embodiment of FIG. 6;

FIG. 9 is a flow chart of control method of a water quality detection system according to an embodiment of the disclosure;

FIG. 10 is a flow chart of control method of a water quality detection system according to another embodiment of the disclosure;

FIG. 11 shows the variation in dissolved oxygen concentration recorded according to the control method of the water quality detection system in the embodiment of FIG. 10; and

FIG. 12 is a chart showing intelligent control of the antibacterial light intensity of the antibacterial light source according to the concentration of the component to be measured according to the embodiment of FIG. 6.

### DETAILED DESCRIPTION

In the following detailed description, for purposes of explanation, numerous specific details are set forth in order to provide a thorough understanding of the disclosed embodiments. According to the description, claims and the drawings disclosed in the specification, one skilled in the art may easily understand the concepts and features of the invention. The following embodiments further illustrate various aspects of the invention, but are not meant to limit the scope of the invention.

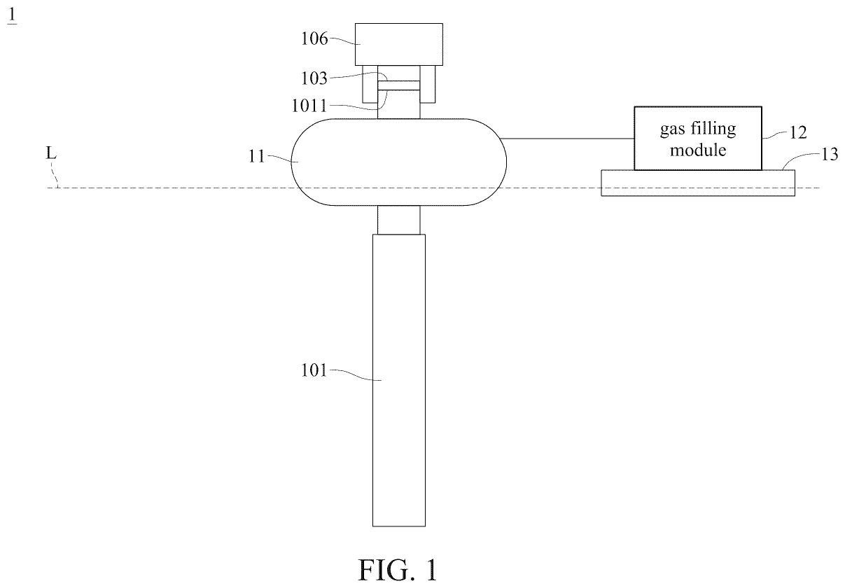

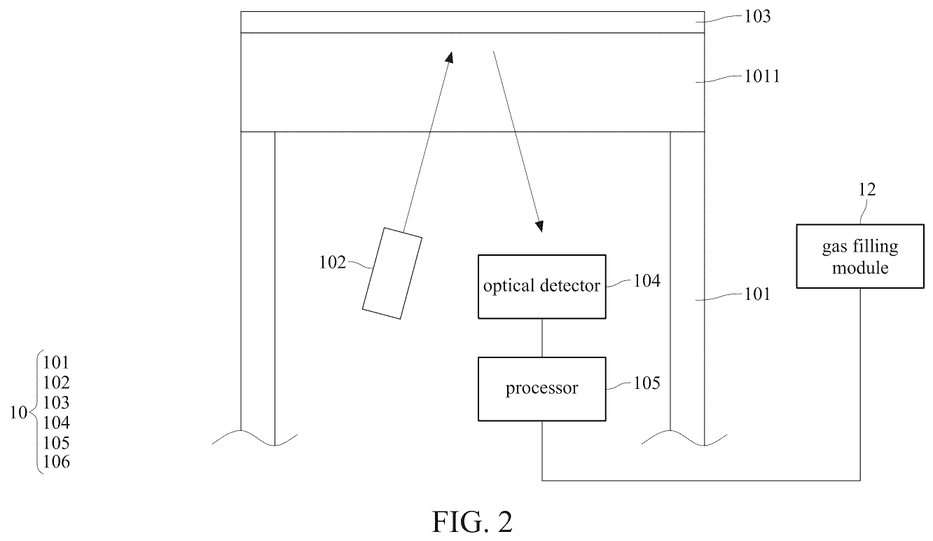

Please refer to FIG. 1 and FIG. 2, FIG. 1 is a schematic diagram of a water quality detection system according to an embodiment of the disclosure, FIG. 2 is a schematic diagram of a water quality detection system according to another embodiment of the disclosure. As shown in FIG. 1 and FIG. 2, a water quality detection system 1, which is configured to measure the concentration of a component to be measured in an aqueous solution, includes a water quality detection device 10, a water-leaving airbag 11, and a gas filling module 12. The water-leaving airbag 11 and the gas filling module 12 are connected to the water quality detection device 10. The water quality detection device 10 includes a body 101, a sensing light source 102, a fluorescent sensing film 103, an optical detector 104 and a processor 105. The sensing light source 102 is disposed in the body 101 and configured to emit sensing light. The fluorescent sensing film 103 is disposed on the surface of the body 101 and configured to receive the sensing light to generate feedback light. The optical detector 104 is disposed in the body 101 and configured to receive the feedback light to generate a concentration signal. The processor 105 is disposed in the body 101 and electrically connected to the optical detector 104, and configured to determine the concentration of the component to be measured in the aqueous solution according to the concentration signal. The water-leaving airbag 11 is disposed at the body 101 and configured to float the fluorescent sensing film 103 to be above the liquid surface L when filled with gas. The gas filling module 12 is configured to inflate or deflate the water-leaving airbag 11.

In this embodiment, the body 101 may have a cavity and a light-transmitting surface. For example, the light-transmitting surface may be located on a light-transmitting substrate 1011 (e.g. a plastic or glass substrate). The body 101 in this embodiment has a rod-shaped structure, but is not limited thereto, that is, the body 101 may also have other arbitrary shapes and structures. The sensing light source 102 may include a reference light source and an excitation light source, wherein the wavelength of the excitation light source may induce excitation of the fluorescent sensing film 103 so that the fluorescent sensing film 103 emits feedback light (fluorescence). Due to the mechanism of fluorescence quenching, the intensity or phase of the excitation light detected by the optical detector may change based on the variation of the concentration of the component to be measured. The wavelength of the reference light source may not produce a fluorescent reaction with the fluorescent sensing film 103, so the intensity or phase of the excitation light detected by the optical detector may not change based on the variation of the concentration of the component to be measured. Based on the difference in the above light intensity or phase signals, the concentration of the aqueous solution may be calculated. For example, the reference light source may be a red light emitting diode, and the excitation light source may be a blue light emitting diode. The fluorescent sensing film 103 may be disposed on the surface of the light-transmitting substrate 1011, and one side of the fluorescent sensing film 103 may be configured to directly contact the aqueous solution to react with the component to be measured in the aqueous solution. When the fluorescent sensing film 103 is excited by the excitation light, it may emit feedback light (fluorescence), and the feedback light may be detected by the optical detector 104. Furthermore, when the fluorescent sensing film 103 reacts with the component to be measured, the signal intensity and phase of the fluorescence it emits may change. In application of detecting dissolved oxygen concentration in aqueous solution, based on the mechanism of fluorescence quenching, the signal intensity or phase of the fluorescence received in the optical detector, which is originated from the emission of the excitation light source toward the fluorescence film, may have a proportional relation with the oxygen concentration in the aqueous solution, while the reference light does not have such relation with the concentration in signal intensity or phase, so the signal intensity difference or the phase difference between the excitation light and the reference light may be used to obtain the oxygen concentration in the aqueous solution.

The optical detector 104 may receive the feedback light and generate a corresponding concentration signal. The optical detector 104 may be, for example, a photodiode, but is not limited thereto. The processor 105 may determine concentration of a component to be measured according to the intensity of the concentration signal when receiving the concentration signal from the optical detector 104. Specifically, the processor 105 may include one or more processing/control units with data receiving, recording, computing, storage and output functions. The processing/control unit is, for example, a microcontroller, a central processing unit, a graphics processor, a programmable logic controller, or any combination of the above. Through the above configuration, when the concentration of the component to be measured in the aqueous solution changes, the signal intensity or phase of the feedback light (fluorescence) may change, and the intensity or phase of the concentration signal may also change, so that the processor 105 may determine the variation of the concentration of the component to be measured in the aqueous solution.

The water-leaving airbag 11 is disposed at the body 101 and configured to float the fluorescent sensing film 103 to be above the liquid surface L when filled with gas. For example, the water-leaving airbag 11 may have a first state and a second state depending on the amount of inflation and deflation, wherein the first state may refer to the water-leaving airbag 11 being fully inflated and the second state may refer to the water-leaving airbag 11 being fully deflated. Regarding the arrangement of the water-leaving airbag 11, there may be various implementation types. Taking the embodiment of FIG. 1 as an example, the water-leaving airbag 11 may be disposed on the side of the body 101 close to the fluorescent sensing film 103. In this way, when the water-leaving airbag 11 is not inflated (in the second state), the fluorescent sensing film 103 may sink below the liquid surface L to measure the concentration of the component to be measured in the aqueous solution; when the water-leaving airbag 11 is inflated (in the first state), the fluorescent sensing film 103 may be floated above the liquid surface L. Furthermore, the water-leaving airbag 11 may be disposed between the center of gravity of the water quality detection device 10 and the fluorescent sensing film 103, so that when the water-leaving airbag 11 is inflated, the heavier side of the water quality detection device 10 (the side where the center of gravity is located) may be kept below the liquid surface L, and the lighter side of the water quality detection device 10 (the side where the fluorescent sensing film 103 is located) may be floated above the liquid surface L. Specifically, the center of gravity may refer to the center of gravity of all components included in the water quality detection device 10. It should be noted that the disclosure is not limited to the aforementioned example of the configuration of the water-leaving airbag 11, any configuration of the water-leaving airbag 11 that can cause the fluorescent sensing film 103 to float above the liquid surface L when inflated can be interpreted as the scope of the disclosure. In practice, the arrangement of the water-leaving airbag 11 may be adjusted according to the shape, structure or weight of the body 101 to achieve the same or similar effect described above.

The gas filling module 12 may be installed on the shore or on a floating platform 13 floating on the liquid surface L, and is connected to the water-leaving airbag 11 with a gas pipeline to inflate or deflate the water-leaving airbag 11. Specifically, the gas filling module 12 may include a pump and/or a controller, and control the inflation or deflation operation of the water-leaving airbag 11 according to a judgment standard or according to the measurement results of the water quality detection device 10. For example, the gas filling module 12 may control the inflating or deflating operation of the water-leaving airbag 11 according to a water-leaving period. Alternatively, the processor 105 may generate a sensing command to drive the gas filling module 12 to deflate the water-leaving airbag 11 so that the fluorescent sensing film 103 sinks below the liquid surface L; and the processor 105 may also generate a water-leaving command to drive the gas filling module 12 to inflate the water-leaving airbag 11, so that the fluorescent sensing film 103 floats above the liquid surface L. In addition, the gas filling module 12 may adjust the length of the water-leaving period according to the concentration of the component to be measured that is measured by the water quality detection device 10 through its own controller (for example, a pump control circuit), to achieve the effect of flexible control. For example, the pump control circuit may have an electrical connection with the processor 105 to obtain the concentration of the component to be measured that is measured by the water quality detection device 10, and determine whether to inflate or deflate the water-leaving airbag 11 according to the concentration of the component to be measured.

Please refer to FIG. 1, the water quality detection system 1 in the embodiment may optionally include a floating platform 13. The floating platform 13 may be equipped with a power supply module, and is electrically connected to the water quality detection device 10 through cables for power supply. Alternatively, the water quality detection device 10 may be equipped with batteries, or may be further equipped with solar panels to generate electricity, which is not limited in the disclosure. Furthermore, the water quality detection device 10 may optionally include a hollow cover 106. In the embodiment, the hollow cover 106 is disposed on the body 101, corresponding to the fluorescent sensing film 103, and an open space allowing the aqueous solution to flow is formed between the hollow cover 106 and the fluorescent sensing film 103 through at least one opening. In view of above, in the embodiment, when the water-leaving airbag 11 is inflated and in the first state, the fluorescent sensing film 103 of the water quality detection device 10 may float above the liquid surface L; when the water-leaving airbag 11 is deflated and in the second state, the fluorescent sensing film 103 of the water quality detection device 10 may sink below the liquid surface L, and is pulled by the air flow pipe between the water-leaving airbag 11 and the gas filling module 12 or the cable between the floating platform 13 and the water quality detection device 10 to be at a given depth below the liquid surface L. In this way, the fluorescent sensing film 103 may be prevented from being in the water to be measured for a long time and breed bacteria, thereby effectively extending the service life of the product.

Furthermore, the water-leaving airbag 11 may, in addition to the first state of fully inflated (100% inflation) and the second state of completely deflated (0% inflation), have multiple intermediate states according to the amount of inflation and deflation. In the embodiment, the gas filling module 12 may further adjust the balance between the buoyancy of the water-leaving airbag 11 and the gravity of the sensor body by controlling the amount of inflation of the water-leaving airbag 11 to be within 0% to 100%, so that the fluorescent sensing film may be suspended at different specific depths below the liquid surface L to meet the needs of detecting different species (for example, fish and shrimp inhabiting at different depths). It is also possible to measure the dissolved oxygen value at different depths through the process of inflating and deflating the water-leaving airbag, and establish a two-dimensional numerical diagram of the change in dissolved oxygen concentration in the vertical direction of the water surface to grasp the distribution of dissolved oxygen in the water. In addition, if there is horizontal water flow interference during the suspension process, the current suspension depth may be confirmed through a gravity sensing chip, such as a three-axis gyroscope or a three-axis accelerometer, and then achieve suspension in water at a specific depth by adjusting the amount of inflation and deflation.

The water-leaving airbag and the gas filling module of the disclosure are combined with the water quality detection device configured to detect dissolved oxygen concentration. However, they can also be combined with detection devices with more or different detection functions, such as a detection device for detecting pH value, so the water quality detection device is not limited to detecting dissolved oxygen concentration. In addition, the water-leaving airbag and the gas filling module of the disclosure may also be combined with a variety of detection devices with different functions, such as a combination of detection devices for detecting dissolved oxygen concentration and pH value, so as to drive multiple detection devices to the liquid surface at the same time.

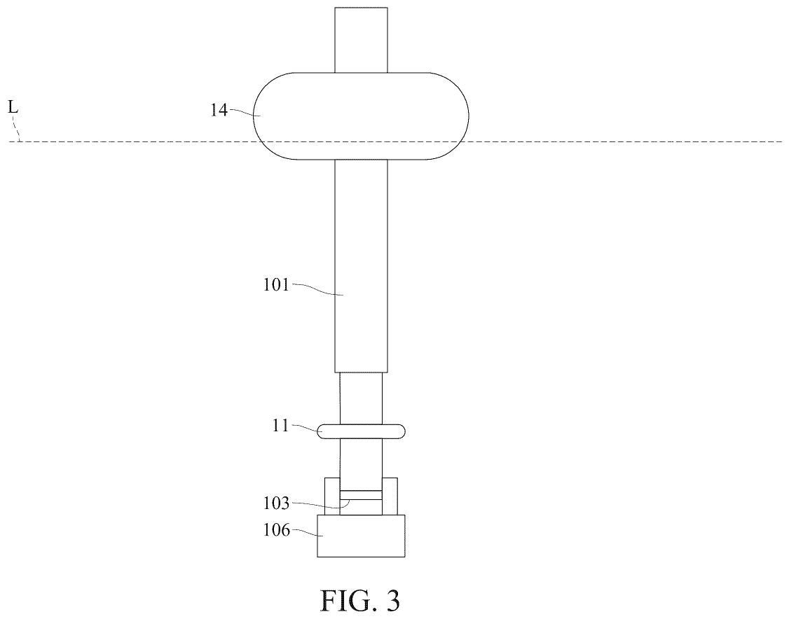

Please refer to FIG. 3 along with FIG. 1, FIG. 3 is a schematic diagram of a water quality detection system in a sensing state according to another embodiment of the disclosure. Compared with the embodiment in FIG. 1, the water quality detection system of the embodiment may further include a floating body 14. Compared to the water-leaving airbag 11 which is disposed on the side of the body 101 close to the fluorescent sensing film 103, the floating body 14 may be disposed on the other side of the body 101 away from the fluorescent sensing film 103. The floating body 14 may provide a buoyancy greater than the overall weight of the water quality detection device. For example, the floating body 14 may be realized through a closed hollow tube made of plastic material, or through a low-density solid object (such as EVA foam), and the shape of the floating body 14 may be but not limited to circular, square, central perforated, etc. Through this configuration, the end of the body 101 provided with the floating body 14 may continuously float above the liquid surface L, and when the water-leaving airbag 11 is inflated, the fluorescent sensing film 103 may float above the liquid surface L, and the body 101 may also completely leave the water surface, thereby reducing the corrosion caused due to the body 101 being immersed in sea water for a long time.

The water-leaving airbag, the gas filling module and the floating body of the disclosure are combined with the water quality detection device configured to detect dissolved oxygen concentration. However, they can also be combined with detection devices with different or more detection functions, such as a detection device for detecting pH value, so the water quality detection device is not limited to detecting dissolved oxygen concentration. In addition, the water-leaving airbag, the gas filling module and the floating body of the disclosure may also be combined with a variety of detection devices with different functions, such as a combination of detection devices for detecting dissolved oxygen concentration and pH value, so as to drive multiple detection devices to the liquid surface at the same time.

According to still another embodiment of the disclosure, the floating body 14 may also be another airbag that is inflated and deflated through the gas filling module 12. When the floating body 14 is inflated and the water-leaving airbag 11 is deflated, the fluorescent sensing film 103 may sink below the liquid surface L and the water quality detection device enters the sensing state; when the floating body 14 is deflated and the water-leaving airbag 11 is inflated, the fluorescent sensing film 103 may float above the liquid surface L. In another embodiment, the floating body 14 and the water-leaving airbag 11 are inflated at the same time, and the fluorescent sensing film 103 may also correspondingly float above the liquid surface L. In comparison, the main difference between the state in which the floating body 14 and the water-leaving airbag 11 are inflated at the same time and the state in which the floating body 14 is deflated and the water-leaving airbag 11 is inflated, is that the body 101 may completely leave the water surface, thereby reducing the corrosion caused due to the body 101 being immersed in sea water for a long time.

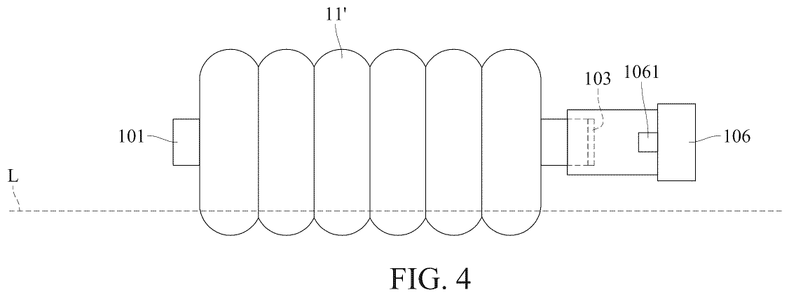

Please refer to FIG. 4 which is a schematic diagram of a water quality detection system according to still another embodiment of the disclosure. In the embodiment, repeated descriptions of the same components as those in the previous embodiment, such as the body 101, the fluorescent sensing film 103, and the hollow cover 106 (including at least one opening 1061), are omitted. In addition, FIG. 4 (and some of the following figures) may appropriately omit the illustration of certain elements (for example, the gas filling module) without obscuring understanding, so as to make the description and illustration more concise. In this embodiment, the water-leaving airbag 11 extends from one side to the other side of the body 101. When the water-leaving airbag 11 is inflated (in the first state), the body 101 together with the fluorescent sensing film 103 may float above the liquid surface L; when the water-leaving airbag is deflated (in the second state), the body 101 together with the fluorescent sensing film 103 may sink below the liquid surface L.

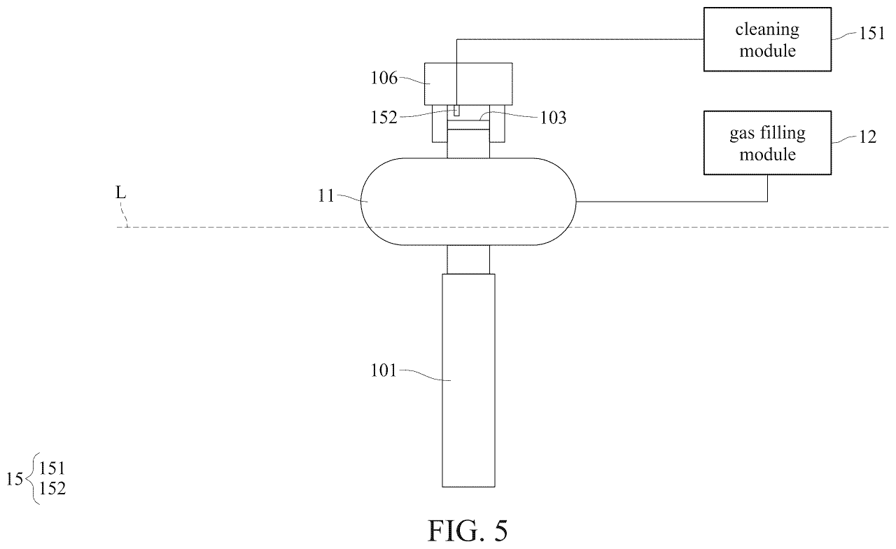

Please refer to FIG. 5 which is a schematic diagram of a water quality detection system according to yet another embodiment of the disclosure. In the embodiment, repeated descriptions of the same components as those in the previous embodiment, such as the body 101, the fluorescent sensing film 103, and the gas filling module 12, is omitted. As shown in FIG. 5, the water quality detection system in this embodiment may further include a cleaning device 15, which is disposed on the hollow cover 106 and corresponds to the fluorescent sensing film 103, and is configured to clean the fluorescent sensing film 103 after it emerges from the liquid surface L. Specifically, the cleaning device 15 may include a cleaning module 151 and a nozzle 152, wherein the cleaning module 151 is connected to the nozzle 152 through a pipeline and uses water flow or air flow to clean the fluorescent sensing film 103. Through this cleaning device 15, when the water-leaving airbag 11 is inflated and the fluorescent sensing film 103 floats above the liquid surface L, foreign object on the surface of the fluorescent sensing film 103 may be cleaned by water flow or air flow, so as to effectively extend the maintenance period of the fluorescent sensing film 103. It should be noted that the cleaning module 151 may have an electrical connection with the gas filling module 12, so that the cleaning module 151 may start the cleaning operation after determining that the water-leaving airbag 11 is inflated and the fluorescent sensing film 103 floats above the liquid surface L.

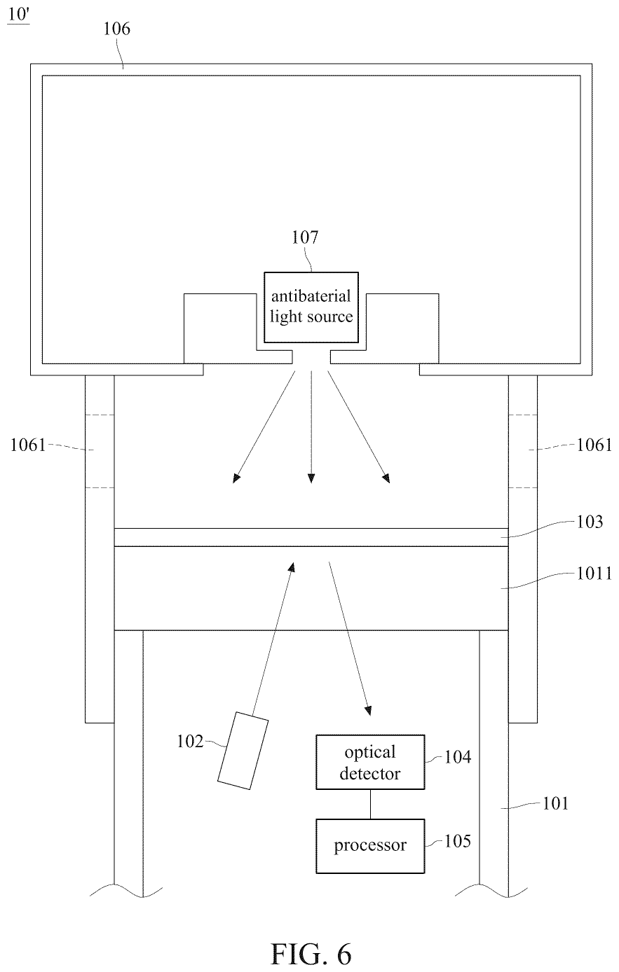

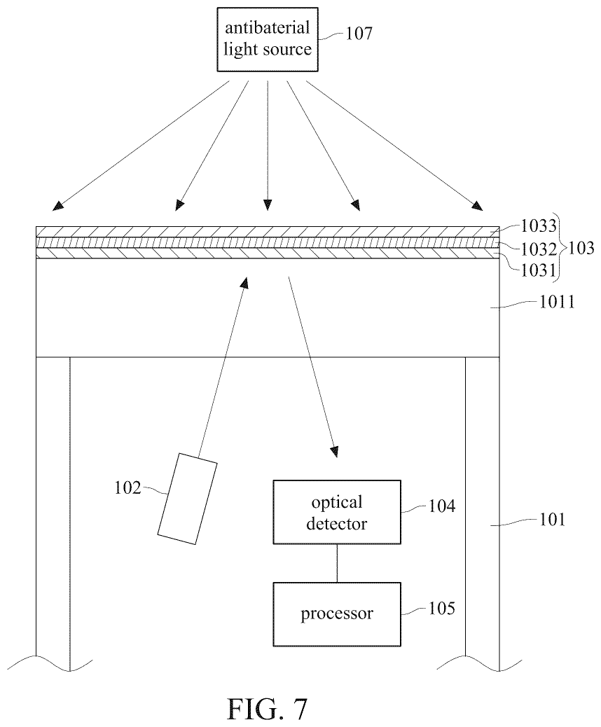

Please refer to FIG. 6 and FIG. 7, FIG. 6 is a schematic diagram of a water quality detection device according to an embodiment of the disclosure, FIG. 7 is another schematic diagram of the water quality detection device according to the embodiment of FIG. 6. As shown in FIG. 6, the water quality detection device 10′ for measuring the concentration of a component to be measured in an aqueous solution includes a body 101, a sensing light source 102, a fluorescent sensing film 103, an optical detector 104, a processor 105 and an antibacterial light source 107. The sensing light source 102 is disposed in the body 101 and configured to emit sensing light. The fluorescent sensing film 103 includes a reaction layer 1031 and a light-shielding layer 1033, the reaction layer 1031 is disposed on a light-transmitting surface of the body 101, the light-shielding layer 1033 is disposed on the reaction layer 1031 and configured to be in direct contact with the aqueous solution, and the reaction layer 1031 is configured to receive the sensing light to generate feedback light. The optical detector 104 is disposed in the body 101 and configured to receive the feedback light to generate a concentration signal. The processor 105 is disposed in the body 101 and electrically connected to the optical detector 104, and configured to determine the concentration of the component to be measured in the aqueous solution according to the concentration signal. The antibacterial light source 107 is disposed at the body and configured to emit antibacterial light to one side of the light-shielding layer 1033 that is in direct contact with the aqueous solution.

Compared with the embodiments of FIG. 1 to FIG. 5, the water quality detection system of this embodiment may not include the water-leaving airbag or the gas filling module, but may additionally include the antibacterial light source 107. Alternatively, the water quality detection system may also include the water-leaving airbag, the gas filling module and the antibacterial light source 107, which is not limited here. Regarding the same components as those in the previous embodiments, such as the body 101, the sensing light source 102, the optical detector 104, etc., repeated descriptions thereof may be omitted. As shown in FIG. 7, in this embodiment, the fluorescent sensing film 103 may have a three-layer structure, specifically including a reaction layer 1031, a reflective layer 1032 and a light-shielding layer 1033. In application of detecting dissolved oxygen in water, the reaction layer 1031 is configured to generate fluorescence and may react with oxygen; the reflective layer 1032 is configured to reflect light and may allow oxygen molecules to permeate; the light-shielding layer 1033 is configured to block light from outside the body 101 (for example, the antibacterial light of the antibacterial light source 107), and may allow oxygen molecules to permeate. Through this stacked structure, the antibacterial light of the antibacterial light source 107 may be irradiated on the side of the light-shielding layer 1033 that is in direct contact with the aqueous solution, thereby reducing the breeding of bacteria, microorganisms, and barnacles, while not interfering with the measurement operation of the optical detector 104 located inside the body 101.

The water quality detection device 10′ may optionally include a hollow cover 106. The hollow cover 106 is disposed on the body 101, corresponding to the fluorescent sensing film 103, and an open space allowing the aqueous solution to flow is formed between the hollow cover 106 and the fluorescent sensing film 103, wherein the antibacterial light source 107 is provided in the hollow cover 106. As shown in FIG. 6, the hollow cover 106 may have a plurality of openings 1061 to form an open space between the hollow cover 106 and the fluorescent sensing film 103. In this embodiment, the antibacterial light source 107 may be disposed on the body 101 through the hollow cover 106. However, in other embodiments, the antibacterial light source 107 may also be disposed inside or outside the body 101, which is not limited in this disclosure. For example, the antibacterial light source 107 may be installed inside the body 101 and guide the antibacterial light to the outside of the body through a special optical guiding design, to perform bacteriostasis on the side of the fluorescent sensing film 103 that is in contact with the aqueous solution, thereby reducing the breeding of bacteria, microorganisms, and barnacles. Specifically, the antibacterial light source 107 may include a light emitting diode with a specific wavelength.

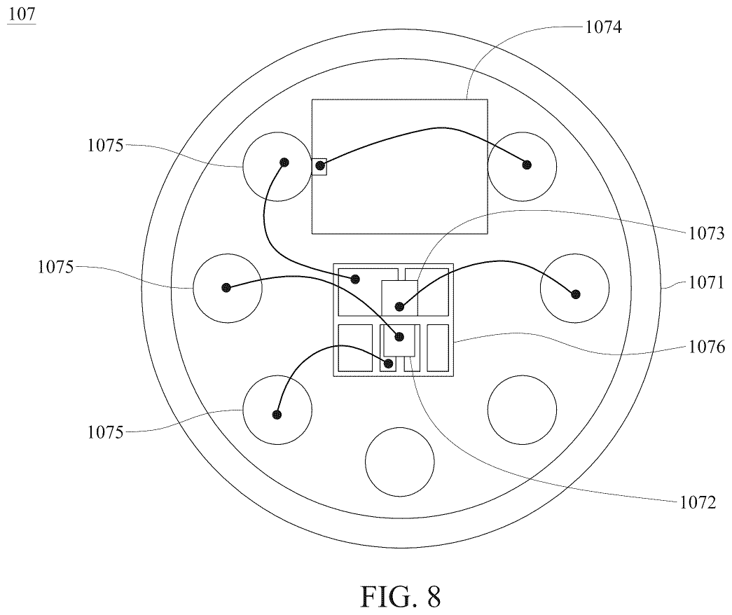

Please refer to FIG. 8 along with FIG. 6 and FIG. 7, FIG. 8 is a schematic diagram of the antibacterial light source of the water quality detection device according to the embodiment of FIG. 6. As an example, the antibacterial light source 107 may be a semiconductor device, such as TO-39 package structure, or surface mount device (SMD). As shown in FIG. 8, the antibacterial light source 107 may include an encapsulation housing 1071, a first light-emitting element 1072, a second light-emitting element 1073, a photoelectric sensing element 1074, a plurality of electrode contacts 1075 and a circuit layer 1076. In this embodiment, the antibacterial light source 107 may emit antibacterial light of blue or ultraviolet wavelength. For example, the first light-emitting element 1072 may be a blue light-emitting diode with a central wavelength of 410 nanometers, and the second light-emitting element 1073 may be an ultraviolet light-emitting diode with a central wavelength of 265 nanometers. By selecting the antibacterial light source 107 with blue and ultraviolet wavelengths, specific bacteria and aquatic organisms may be inhibited. For example, the blue light with a wavelength of 410 nanometers may inhibit the attachment and growth of aquatic organisms (such as barnacles), and the ultraviolet light with a wavelength of 265 nanometers may inhibit the attachment and growth of bacteria (biofilms). The photoelectric sensing element 1074 may be, for example, a photodiode, configured to sense the lateral light emitted by the first light-emitting element 1072 and the second light-emitting element 1073, and thereby monitor the intensity of the antibacterial light. The driving current of the two light-emitting elements may be controlled through a feedback circuit to achieve stable control of the antibacterial light intensity. In addition, the state of decline and lifespan of the antibacterial light source may be estimated based on changes in the driving current. It should be noted that for the active biological species in the use environment, those with ordinary knowledge in the art may use light-emitting elements of any wavelength combined as the antibacterial light source in this disclosure. For example, the wavelength of the antibacterial light source may be within 250 and 285 nanometers, but is not limited thereto.

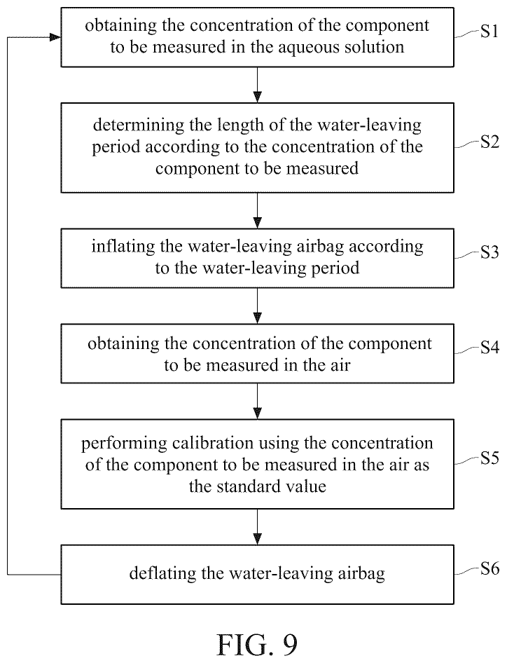

The water quality detection devices of the above embodiments may be combined with each other to produce additive effects. The operation of the water quality detection device of each embodiment may be further explained below. Please refer to FIG. 9 which is a flow chart of control method of a water quality detection system according to an embodiment of the disclosure. As shown in FIG. 9, the water quality detection system may perform the following processes to continuously measure the concentration of the component to be measured in the aqueous solution, including step S1: obtaining the concentration of the component to be measured in the aqueous solution; step S2: determining the length of the water-leaving period according to the concentration of the component to be measured; step S3: inflating the water-leaving airbag according to the water-leaving period; step S4: obtaining the concentration of the component to be measured in the air; step S5: performing calibration using the concentration of the component to be measured in the air as the standard value; and step S6: deflating the water-leaving airbag, and then returns to step S1. In this embodiment, steps S2, S4, and S5 are optional. For example, in step S3, the processor may control the gas filling module to inflate the water-leaving airbag to be in the first state according to a given water-leaving period. In step S6, the processor may control the gas filling module to deflate the water-leaving airbag to be in the second state after meeting the set water-leaving period time length to complete a water-leaving-submersing period operation. In addition, as shown in the embodiment of FIG. 3, the processor may also control the gas filling module to switch the inflating or deflating of the water-leaving airbag and the floating body according to the water-leaving period. That is, when the gas filling module deflates the water-leaving airbag, it may also inflate the floating body at the same time. Through this water-leaving solution, the time for the fluorescent sensing film and body to sink underwater may be reduced, thereby effectively extending the maintenance period of the sensor, reducing maintenance costs, and extending the service life of the sensor.

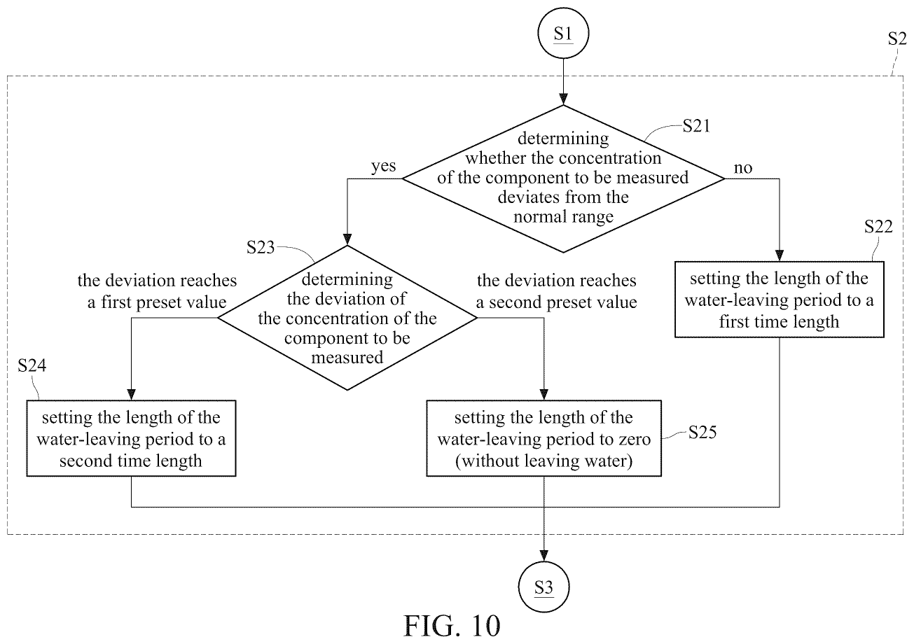

The intelligent water-leaving control scheme of step S2 is described below. Please refer to FIG. 1 and FIG. 2, in this embodiment, the processor 105 may be electrically connected to the gas filling module 12, and the processor 105 may be further configured to determine a water-leaving period according to the concentration of the component to be measured, and control the gas filling module 12 to inflate or deflate the water-leaving airbag 11 according to the water-leaving period. As described above, the gas filling module 12 may be installed on the shore or on a floating platform, and an electrical connection may be established between the processor 105 and the gas filling module 12 through a specific cable or wireless transmission method to achieve signal communication between above and below the water surface. In step S2, the processor 105 may determine the length of the water-leaving period according to the concentration of the component to be measured. Specifically, as the concentration of the component to be measured is higher, the length of the water-leaving period may be longer. Please refer to FIG. 10 which is a flow chart of control method of a water quality detection system according to another embodiment of the disclosure. As shown in FIG. 10, for example, step S2 may include step S21: determining whether the concentration of the component to be measured deviates from the normal range; if the determination result in step S21 is no, step S22 is executed: setting the length of the water-leaving period to a first time length; if the determination result in step S21 is yes, step S23 is executed: determining the deviation of the concentration of the component to be measured; when the determination result in step S23 corresponds to the deviation reaching a first preset value, step S24 is executed: setting the length of the water-leaving period to a second time length; when the determination result in step S23 corresponds to the deviation reaching a second preset value, step S25 is executed: setting the length of the water-leaving period to zero (not leaving water); and after steps S22, S24, and S25, step S3 shown in FIG. 9 is executed. The second preset value is greater than the first preset value, and the first time length is greater than the second time length. In addition, the normal range may be input into the system in advance, or may be determined based on historical measurement data.

In application of detecting dissolved oxygen in water, in steps S21 and S22, when the dissolved oxygen concentration obtained by the processor does not deviate from the normal range of dissolved oxygen concentration by 20%, the processor may set the length of the water-leaving period to a longer first time length (for example, 60 minutes), so that the fluorescent sensing film is to sink under the liquid surface once every 60 minutes, and each sinking duration may be 5 to 10 minutes (depending on the stabilization time of the sensing signal) to perform periodic measurements. In steps S23 and S24, when the dissolved oxygen concentration obtained by the processor deviates from the normal range of dissolved oxygen concentration by 20%, the processor may set the length of the water-leaving period to a shorter second time length (for example, 30 minutes), so that the fluorescent sensing film is to sink under the liquid surface once every 30 minutes to perform periodic measurements. In steps S23 and S25, when the dissolved oxygen concentration obtained by the processor deviates from the normal range of dissolved oxygen concentration by 40%, the processor may set the length of the water-leaving period to zero, so that the fluorescent sensing film is sinking below the liquid surface without leaving the water for continuous measurement. It can be understood that the criteria for determining the deviation of component concentration may be defined with more intermediate intervals, and different water-leaving periods may be selected for each interval.

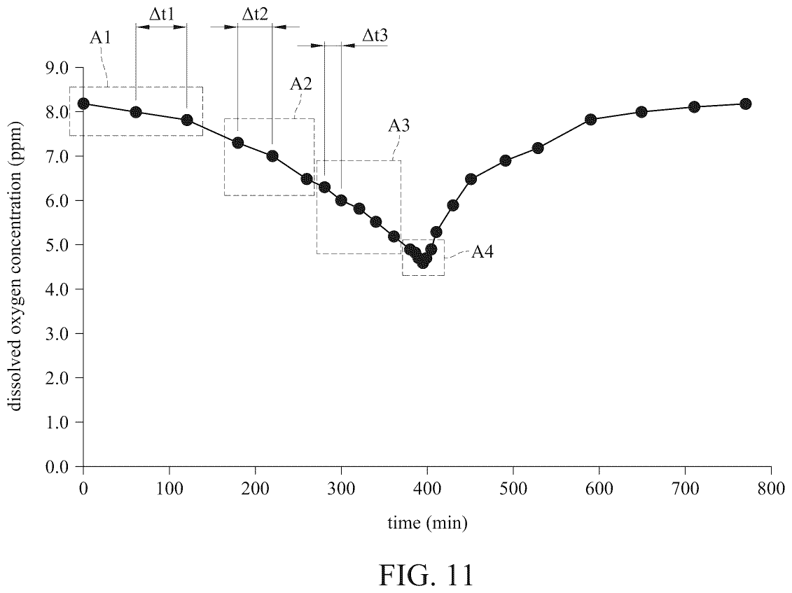

Please refer to FIG. 11 which shows the variation in dissolved oxygen concentration recorded according to the control method of the water quality detection system in the embodiment of FIG. 10. As shown in FIG. 11, in application of detecting dissolved oxygen in water, the normal range of dissolved oxygen concentration in water may be defined in two ways. The first scheme is based on past measurement experience of which the criteria may be adjusted according to time. For example, in a fish farming environment, photosynthesis and oxygen consumption are different at day and night. The dissolved oxygen concentration>8 ppm at noon is within the normal range, and the dissolved oxygen concentration>4 ppm in the morning is within the normal range. The second scheme is that the normal range can be determined based on single criteria. For example, concentration>8 ppm is the normal range, and the concentration<4 ppm is the dangerous range. In the first scheme, the normal range of dissolved oxygen concentration is different at day and night, and the normal range in the following example is 8 ppm. In interval A1, the measured dissolved oxygen concentration is around 8 ppm, which does not deviate from the normal range by 10%, at this time, the longest water-leaving period (for example, 60 minutes/time) may be used for measurement, that is, Δt1 is 60 minutes; in interval A2, the measured dissolved oxygen concentration drops to around 7 ppm, which deviates from the normal range by 10%, at this time, shorter water-leaving period (for example, 40 minutes/time) may be used for measurement, that is, Δt2 is 40 minutes; in interval A3, the measured dissolved oxygen concentration drops to around 5-6 ppm, which deviates from the normal range by 20%, at this time, even shorter water-leaving period (for example, 20 minutes/time) may be used for measurement, that is, Δt3 is 20 minutes; in interval A4, the measured dissolved oxygen concentration drops to below 5 ppm, which deviates from the normal range by 40%, at this time, the water-leaving period may be set to zero for continuous measurement. In the second scheme which uses single criteria for determining the normal range of the dissolved oxygen concentration, if the dissolved oxygen concentration is higher than twice of the normal value (assuming 4 ppm), for example, the dissolved oxygen concentration>8 ppm, the water-leaving period is set to T1 (for example, 60 minutes); if the dissolved oxygen concentration is within 1 and 2 times of the normal value, for example, 4-8 ppm, the water-leaving period is set to T2 (for example, 30 minutes); if the dissolved oxygen concentration is lower than the normal value, for example, the dissolved oxygen concentration<4 ppm, the water-leaving operation is not performed.

Through the intelligent water-leaving control scheme, the time for the fluorescent sensing film sinking under the liquid surface may be reduced when the water quality is relatively stable, to effectively extend the maintenance cycle of the sensor, reduce maintenance costs and increase the service life of the sensor; and when the water quality is poor, the time for the fluorescent sensing film sinking under the liquid surface is increased (or even without leaving the water) to monitor changes in water quality data in a detailed way.

The water-leaving calibration control scheme of steps S4 and S5 is described below. When the water-leaving airbag is inflated (in the first state) and the fluorescent sensing film floats above the liquid surface, the processor may obtain the concentration of the component to be measured in the air, and perform calibration using the concentration of the component to be measured in the air as the standard value. In application of detecting dissolved oxygen, the processor may perform calibration based on the oxygen concentration in the air. Specifically, the processor determines the dissolved oxygen concentration based on the concentration signal measured by the optical detector, so when the fluorescent sensing film is exposed to the air, the processor may determine the oxygen concentration in the air based on the concentration signal measured by the optical detector, and use it as a reference value (dissolved oxygen concentration is 100%). Afterwards, when the fluorescent sensing film sinks below the liquid surface, the processor can calibrate the dissolved oxygen concentration in the aqueous solution based on the updated baseline value of dissolved oxygen concentration.

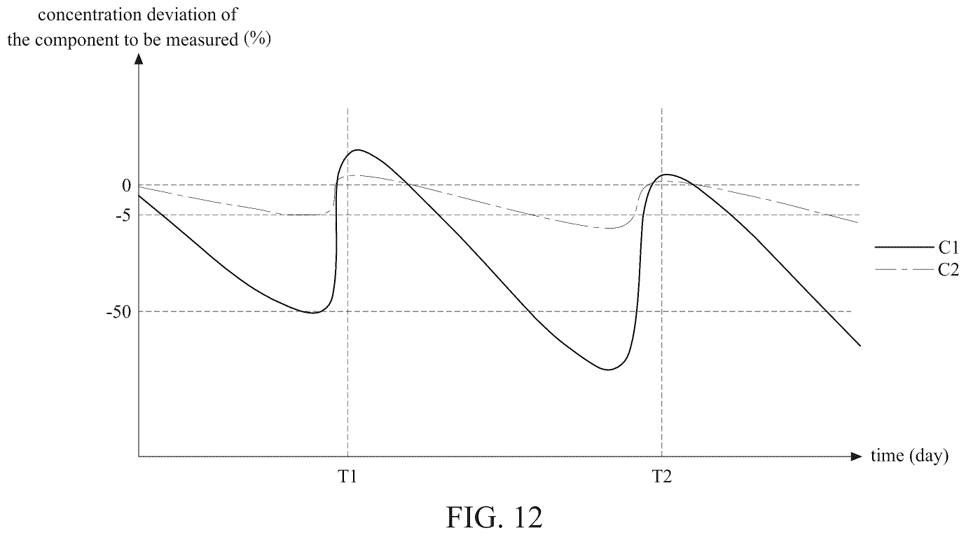

In the embodiment of FIG. 6, the antibacterial light source 107 can emit antibacterial light and determine the intensity of the antibacterial light through control executed by a processor of its own control circuit. Alternatively, the antibacterial light source 107 may be electrically connected to the processor 105, and the processor 105 may be further configured to control the intensity of the antibacterial light of the antibacterial light source 107 according to the concentration of the component to be measured. For example, the antibacterial light source 107 may include a light emitting control unit, and the processor 105 and the antibacterial light source 107 may be electrically connected through a specific cable, wherein this cable may pass through the hole in the body 101 to electrically connect the processor 105 and the antibacterial light source 107. Please refer to FIG. 12, FIG. 12 is a chart showing intelligent control of the antibacterial light intensity of the antibacterial light source according to the concentration of the component to be measured according to the embodiment of FIG. 6. As shown in FIG. 12, in the interval from time 0 to T1, the concentration deviation of the component to be measured in data C1 reaches −50%, at this time, the processor may control the antibacterial light source to increase the intensity of the antibacterial light; in the interval from time T1 to T2, the concentration deviation of the component to be measured in data C2 merely reaches −5%, at this time, the processor may control the antibacterial light source to increase the intensity of the antibacterial light. By controlling the antibacterial light source to intelligently regulate the intensity of the antibacterial light, it is possible to strengthen the irradiation of the antibacterial light when the measurement signal deviation is large, increasing the antibacterial effect and maintain measurement accuracy; and when the measurement signal deviation is small, the irradiation of the antibacterial light is weakened, thereby saving system energy consumption and extending the service life of the antibacterial light source. It should be noted that although this embodiment describes using the processor 105 to control the antibacterial light intensity of the antibacterial light source 107, in practice, the antibacterial light source 107 may have its own light-emitting control unit which is configured to adjust the anti-bacterial light intensity, so this disclosure is not limited thereto.

In view of the above description, the water quality detection system and water quality detection device disclosed in this disclosure, by disposing the water-leaving airbag on the body of the water quality detection device, may make the fluorescent sensing film float above the liquid surface when the water-leaving airbag is inflated, reduce the time that the fluorescent sensing film continues to be immersed in the water to avoid the fluorescent sensing film to be rapidly worn out. On the other hand, by disposing the antibacterial light source, the water quality detection device may emit antibacterial light to the side of the fluorescent sensing film that is in contact with the water, thereby inhibiting the attachment of barnacles or other microorganisms in the water to avoid inaccurate measurement of the fluorescent sensing film. Therefore, the water quality detection device in this disclosure may effectively extend the maintenance cycle of the sensor, reduce maintenance costs, and increase the service life of the sensor. In addition, through the intelligent scheme of controlling the water-leaving period, when the water quality is relatively stable, the time for the fluorescent sensing film to sink below the liquid surface may be reduced, thereby reducing maintenance costs; and when the water quality is poor, the time for the fluorescent sensing film to sink under the liquid surface is increased (or even without leaving the water), thereby monitoring changes in water quality data in a detailed way. Through intelligent control of antibacterial light intensity, the antibacterial light irradiation may be strengthened to maintain measurement accuracy when the measurement signal deviation is large; and when the measurement signal deviation is small, the irradiation of the antibacterial light is weakened to extend the service life of the antibacterial light source.

What is claimed is:

1. A water quality detection system, configured to measure a concentration of a component to be measured in an aqueous solution, the water quality detection system comprising a water quality detection device and a water-leaving airbag and a gas filling module connected to the water quality detection device, the water quality detection device comprising:
a body;
a sensing light source disposed in the body and configured to emit sensing light;
a fluorescent sensing film disposed on a surface of the body and configured to receive the sensing light to generate feedback light;
an optical detector disposed in the body and configured to receive the feedback light to generate a concentration signal; and
a processor disposed in the body and electrically connected to the optical detector, and configured to determine the concentration of the component to be measured in the aqueous solution according to the concentration signal,
wherein the gas filling module is configured to inflate or deflate the water-leaving airbag, and the water-leaving airbag is disposed at the body and configured to float the fluorescent sensing film to be above liquid surface when filled with gas.

2. The water quality detection system of claim 1, wherein the water-leaving airbag is disposed on one side of the body close to the fluorescent sensing film.

3. The water quality detection system of claim 1, wherein the water-leaving airbag is disposed between a center of gravity of the water quality detection device and the fluorescent sensing film.

4. The water quality detection system of claim 1, wherein the water-leaving airbag extends from one side to another side of the body.

5. The water quality detection system of claim 1, wherein the processor is electrically connected to the gas filling module, and the processor is further configured to determine a water-leaving period according to the concentration of the component to be measured, and control the gas filling module to inflate or deflate the water-leaving airbag according to the water-leaving period.

6. The water quality detection system of claim 5, wherein the processor is configured to set a length of the water-leaving period to a first time length when determining that the concentration of the component to be measured is within a normal range,
set the length of the water-leaving period to a second time length when determining that a deviation of the concentration of the component to be measured from the normal range reaches a first preset value, and
set the length of the water-leaving period to zero when determining that the deviation of the concentration of the component to be measured from the normal range reaches a second preset value,
wherein the second preset value is greater than the first preset value, and the first time length is greater than the second time length.

7. The water quality detection system of claim 1, wherein the water-leaving airbag has a first state and a second state depending on an amount of inflation and deflation, and the processor is electrically connected to the gas filling module and further configured to perform calibration according to the concentration signal when controlling the gas filling module to inflate the water-leaving airbag to be the first state.

8. The water quality detection system of claim 1, further comprising a floating body disposed on one side of the body away from the fluorescent sensing film.

9. The water quality detection system of claim 1, wherein the water-leaving airbag has a plurality of states depending on an amount of inflation and deflation, and the gas filling module is further configured to control an inflation amount of the water-leaving airbag to make the fluorescent sensing film suspend at different depths below the liquid surface.

10. The water quality detection system of claim 1, further comprising:
a floating platform located on the liquid surface, wherein the gas filling module is disposed on the floating platform.

11. The water quality detection system of claim 1, wherein the water quality detection device further comprises:
a hollow cover disposed on the body, corresponding to the fluorescent sensing film, and an open space allowing the aqueous solution to flow is formed between the hollow cover and the fluorescent sensing film, and
the water quality detection system further comprising:
a cleaning device disposed on the hollow cover, and configured to clean the fluorescent sensing film after the fluorescent sensing film emerging from the liquid surface.

12. The water quality detection system of claim 1, wherein the water quality detection device further comprises:
an antibacterial light source disposed at the body and configured to emit antibacterial light to one side of the fluorescent sensing film that is in direct contact with the aqueous solution.

13. The water quality detection system of claim 12, wherein the antibacterial light emitted by the antibacterial light source comprises blue light or ultraviolet light.

14. The water quality detection system of claim 12, wherein the antibacterial light source comprises a plurality of light-emitting diodes corresponding to different wavelength ranges.

15. The water quality detection system of claim 12, wherein the antibacterial light source is connected to the processor, and the processor is further configured to control intensity of the antibacterial light generated by the antibacterial light source according to the concentration of the component to be measured.

16. A water quality detection device, configured to measure a concentration of a component to be measured in an aqueous solution, the water quality detection device comprising:
a body;
a sensing light source disposed in the body and configured to emit sensing light;
a fluorescent sensing film comprising a reaction layer and a light-shielding layer, the reaction layer disposed on a light-transmitting surface of the body, the light-shielding layer disposed on the reaction layer and configured to be in direct contact with the aqueous solution, and the reaction layer configured to receive the sensing light to generate feedback light;
an optical detector disposed in the body and configured to receive the feedback light to generate a concentration signal;
a processor disposed in the body and electrically connected to the optical detector, and configured to determine the concentration of the component to be measured in the aqueous solution according to the concentration signal; and
an antibacterial light source disposed at the body and configured to emit antibacterial light to one side of the light-shielding layer that is in direct contact with the aqueous solution.

17. The water quality detection device of claim 16, wherein the antibacterial light emitted by the antibacterial light source comprises blue light or ultraviolet light.

18. The water quality detection device of claim 16, wherein the antibacterial light source comprises a plurality of light-emitting diodes corresponding to different wavelength ranges.

19. The water quality detection device of claim 16, wherein the antibacterial light source is connected to the processor, and the processor is further configured to control intensity of the antibacterial light generated by the antibacterial light source according to the concentration of the component to be measured.

20. The water quality detection device of claim 16, further comprising:
a hollow cover disposed on the body, corresponding to the fluorescent sensing film, and an open space allowing the aqueous solution to flow is formed between the hollow cover and the fluorescent sensing film, and the antibacterial light source is disposed on the hollow cover.
